(19) United States
(12) Patent Application Publication
(10) Pub. No.: US 2022/0145237 A1
(43) Pub. Date: May 12, 2022

(54) OPTIMIZED METHOD FOR INDUSTRIAL EXPLOITATION OF UNICELLULAR RED ALGAE

(71) Applicant: FERMENTALG, Libourne (FR)

(72) Inventors: Olivier CAGNAC, Libourne (FR); Axel ATHANE, Carbon Blanc (FR); Julien DEMOL, Les Billaux (FR); Sandra BROSSET-VINCENT, Castres-Gironde (FR)

(21) Appl. No.: 17/427,834

(22) PCT Filed: Feb. 7, 2020
(86) PCT No.: PCT/EP2020/053081

§ 371 Date: Aug. 2, 2021

(30) Foreign Application Priority Data
Feb. 8, 2019 (FR) 1901259

(51) Int. Cl. C12N 1/12; C07K 14/405; C07K 14/795; C12N 1/06; C12P 17/18; A23K 10/16; A23L 17/60; A23L 33/195

(52) U.S. Cl. CPC C12N 1/12; C07K 14/405; C07K 14/795; A23L 33/195; C12P 17/182; A23K 10/16; A23L 17/60; C12N 1/066

(57) ABSTRACT

The present invention relates to a process for the cultivation of unicellular red algae (URA) optimized for the valorization of the culture products, both the biomass obtained, the phycocyanins extracted therefrom or other culture products such as porphyrins or protein extracts.

### FIELD OF THE INVENTION

The present invention relates to a process for the cultivation of unicellular red algae (URA) optimized for the valorization of the culture products, both the biomass obtained, the phycocyanins extracted therefrom or other culture products such as porphyrins or protein extracts.

### PRIOR ART

Different ways of cultivating microalgae, in particular unicellular red algae (URA), are known for the manufacture of different products for industrial or food use.

In particular, mention may be made of processes for cultivating and processing spirulina for the manufacture of protein-rich food supplements or for the manufacture of phycocyanins useful as food coloring.

Mention may also be made of processes for cultivating and processing URA to obtain similar products and in particular processes for cultivating and processing Galdieria sulphuraria described in patent applications WO 2017/050917, WO 2017/093345 and WO 2018/178334.

An industrial process for cultivating and processing URA such as Galdieria sulphuraria is shown in FIG. 1.

It should be noted, however, that these different processes can be further improved at each step of the production and processing of URA. In particular, these processes can be improved in view of the fact that URA such as Galdieria sulphuraria accumulate large amounts of glycogen when cultivated under usual culture conditions, amounts of glycogen which have an impact in particular on the conditions of treatment of the biomass, in particular cell lysis, the conditions of isolation of the products from the biomass and the quality of these isolated products, in particular the phycocyanins.

In particular, it is important to be able to optimize these various steps to better valorize the products obtained.

### DISCLOSURE OF THE INVENTION

The invention relates to an optimized process for the cultivation and valorization of URA, in particular of Galdieria sulphuraria, comprising steps (a) of fermentation culture of the URA, (b) of separation of the biomass from the fermentation juice, if need be (c) of cell lysis and if need be a step (d) of extraction of valorizable products from the lysed biomass, which comprises at least one of the following steps of:

(c1) lysis by grinding while maintaining the biomass during grinding at a temperature below 50° C. and/or

(a1) fermentation culture with a maturation phase with limited carbon source input into the culture medium, and/or

(b1) extraction of porphyrins from the fermentation juice, and/or

(d1) extraction of phycocyanins from the lysed biomass by at least 2 successive washes in amounts of water representing in total less than 4 times the total volume of lysed biomass.

The invention also relates to the products obtained by the process, in particular the porphyrins extracted from the fermentation juice, the biomass, the lysed biomass, the isolated proteins and/or phycocyanins.

### DESCRIPTION OF THE FIGURES

FIG. 1 represents a simplified diagram of the process for manufacturing different products from the Galdieria sulphuraria culture.

FIG. 2 represents the growth of the Galdieria sulphuraria strain in fed-batch mode on glycerol with maturation phase.

FIG. 3 represents the monitoring of the biomass composition during the fed-batch culture on glycerol with maturation phase.

FIG. 4 represents the growth of the Galdieria sulphuraria strain in fed-batch mode on milk permeate with maturation phase.

FIG. 5 represents the monitoring of biomass composition during the culture on milk permeate in fed-batch mode with maturation phase.

FIG. 6 represents the growth monitoring of a Galdieria sulphuraria strain grown continuously on glycerol without porphyrin production.

FIG. 7 represents the monitoring of the biomass composition during the culture of Galdieria sulphuraria grown continuously on glycerol.

FIG. 8 represents the growth monitoring of a Galdieria sulphuraria strain in continuous mode on glucose.

FIG. 9 represents the monitoring of biomass composition during the culture of Galdieria sulphuraria grown continuously on glucose.

FIG. 10 represents the growth monitoring of a Galdieria sulphuraria strain in continuous mode on milk permeate.

FIG. 11 represents the monitoring of the biomass composition during the culture of Galdieria sulphuraria grown continuously on milk permeate.

FIG. 12 shows the Bertoli HHP grinding data (1200 bar) without cooling.

FIG. 13 shows the Bertoli HHP grinding data (1200 bar) with cooling.

FIG. 14 represents the resistance of phycocyanin at 50° C. on lysates adjusted to different pH.

FIG. 15 represents the effect of bead diameter on the rate of cell lysis by ball mill.

FIG. 16 represents the amounts of phycocyanins extracted with serial washes compared with single washes for different volumes of water.

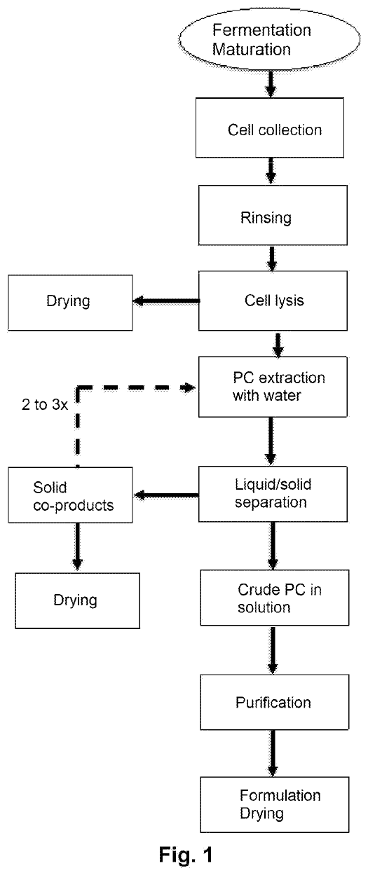

### DETAILED DESCRIPTION OF THE INVENTION

The various steps of the process in accordance with the invention are described in greater detail below and in the examples.

According to the invention, “valorization” means the technical steps that allow the isolation of useful products for use in industry.

In particular, the process in accordance with the invention comprises the following successive steps:

(a1) of fermentation culture with a maturation phase which includes the limitation of the carbon source in the culture medium,

(b1) if need be, of extraction of porphyrins from the fermentation juice,

(c1) if need be, of lysis by grinding while maintaining the biomass during grinding at a temperature below 50° C. and,

(d1) if need be, of extraction of phycocyanins from the lysed biomass by at least 2 successive washes in amounts of water totaling less than 4 times the total volume of lysed biomass.

More particularly, the process in accordance with the invention comprises the following successive steps:

(a1) of fermentation culture with a maturation phase which includes the limitation of the carbon source in the culture medium,

(b1) of extraction of porphyrins from the fermentation juice,

(c1) of lysis by grinding while maintaining the biomass during grinding at a temperature below 50° C. and,

(d1) of extraction of phycocyanins from the lysed biomass by successive washes in amounts of water totaling less than 3 times the total volume of lysed biomass.

According to a particular embodiment of the invention, the process comprises at least the following steps:

(a1) of fermentation culture with a maturation phase by limiting the supply of carbon source in the culture medium, and

(b1) of extraction of porphyrins from the fermentation juice.

According to another particular embodiment of the invention, the process comprises at least the following steps:

(c1) of lysis by grinding while maintaining the biomass during grinding at a temperature below 50° C. and, if need be

(d1) of extraction of phycocyanins from the lysed biomass by successive washes in amounts of water totaling less than 3 times the total volume of lysed biomass.

URA are well known to the person skilled in the art, in particular URA that can be cultivated industrially for the production of biomass and its by-products, proteins or phycocyanins. Particular mention may be made of the algae (or microalgae) of the orders Cyanidiales. The order Cyanidiales includes the families Cyanidiaceae and Galdieriaceae, themselves subdivided into the genera Cyanidioschyzon, Cyanidium and Galdieria, to which belong, inter alia, the species Cyanidioschyzon merolae 10D, Cyanidioschyzon merolae DBV201, Cyanidium caldarum, Cyanidium daedalum, Cyanidium maximum, Cyanidium partitum, Cyanidium rumpens, Galdieria daedala, Galdieria maxima, Galdieria partita and Galdieria sulphuraria. Particular mention may be made of the strain Galdieria sulphuraria (also called Cyanidium caldarium) UTEX 2919.

Mention may also be made of known producers of phycocyanins such as the filamentous cyanobacteria of the genus Arthrospira, which are industrially cultivated under the common name of spirulina.

The microorganisms that produce phycocyanin with a high glycogen content are particularly identified among the microorganisms mentioned above, in particular species of the genera Arthrospira, Spirulina, Synechococcus, Cyanidioschyzon, Cyanidium or Galdieria, in particular Galdieria sulphuraria.

The invention also relates to the products obtained by the process, in particular the biomass, the porphyrins isolated from the fermentation juice, the lysed biomass, the proteins and the phycocyanins isolated from the lysed biomass.

Phycocyanins (PC) produced by microorganisms include c-phycocyanins (C-PC) and allophycocyanins. According to the invention, phycocyanins are defined as C-PCs and allophycocyanins, isolated or mixtures thereof in any proportion, in particular C-PCs.

The Culture of URA (a1).

The fermenter culture of URA and in particular of Galdieria sulphuraria has been widely described in the scientific literature, either in heterotrophic or mixotrophic mode, in fed-batch or continuous mode.

The biomass produced includes not only phycocyanins and proteins, but also reserve sugars like glycogen. Glycogen contents in the biomass are in the order of 20 to 50% by mass in relation to the total mass of dry matter. The higher the glycogen content in the final biomass, the lower the concentration of phycocyanin (PC) and protein. The glycogen produced by URA, in particular in Galdieria, is soluble in cold water and is therefore found in the aqueous phase during the extraction of the PC, which poses technical problems during filtration, such as an increase in viscosity, clogging of the filtration membranes, pressure build-up, accumulation of glycogen in the fraction containing the phycocyanin and thus the obtaining of a less pure phycocyanin. The invention allows, by a “piloting” of fermentation, to reduce the glycogen levels to values lower than 20% in mass/DM.

The invention therefore relates to a process for the production of biomass in accordance with the invention which comprises the fermentation culture of URA as defined above with a maturation phase which comprises limiting the supply of carbon source in the culture medium.

Cultures by fermentation are carried out on a culture medium comprising various nutrients allowing cell growth. These culture media, well known to the person skilled in the art, include a carbon source, a nitrogen source, a phosphorus source, macroelements, microelements, in appropriate concentrations to allow cell growth.

The maturation step is particularly implemented in fed-batch or continuous culture modes.

The carbon source can be any carbon source known to the skilled person and which can be used for the cultivation of URA and in particular Galdieria sulphuraria, such as polyols, in particular glycerol, sugars, such as glucose or sucrose or also lactose or complex media comprising lactose such as milk permeate, serum permeate, buttermilk and mixtures thereof and in particular milk permeate.

It is known that some carbonaceous substrates such as glucose strongly inhibit PC synthesis while others less so. However, for the viability of an industrial PC production process, many economic parameters have to be taken into account, and in particular the cost of raw materials. Thus, the use of a carbon source such as milk permeate containing lactose does not allow PC yields as high as with glycerol, but the overall economy of the process with the use of milk permeate remains advantageous because it is a by-product of the dairy industry that is difficult to recycle. It is all the more advantageous as the implementation of the process in accordance with the invention makes it possible to obtain a high cell density with a low glycogen content, which facilitates the extraction of the PC at the end of the process.

The biomass obtained after maturation has the following composition:

| | Composition in % of dry matter | | |
|---|---|---|---|
| Matured biomass | Protein | glycogen | C-PC |
| Target biomass | >45% | <20% | >1.5% |
| Preferred biomass | 55% to 70% | 5% to 20% | 5 to 10% |

The specific conditions for the implementation of the maturation phase by limiting the carbon source for these two cultivation methods are specified below.

Batch/Fed-Batch Culture.

In a fed-batch culture, the fermentation culture process in accordance with the invention comprises a first phase of cell growth in a culture medium comprising a carbon source as defined above, so as to obtain a cell density in the culture medium of at least 30 g/L DM. The person skilled in the art will know how to define the composition of the culture media suitable for obtaining such a cell density and in particular the carbon source content, in particular with regard to the processes of the prior art described in particular in patent applications WO 2017/050917, WO 2017/093345 and WO 2018/178334.

Once the desired cell density has been obtained, a “maturation” phase is carried out, which consists of weaning the strain off the organic carbon substrate.

According to a particular embodiment of the invention, the maturation phase is triggered once the culture in the growth phase reaches at least 30 g/L DM, preferably at least 80 g/L DM, more preferentially at least 100 g/L dry matter.

During the fed-batch phase, the culture is fed with a feeding medium comprising at least 100 g/L carbon substrate, preferably at least 200 g/L carbon substrate, more preferentially at least 500 g/L carbon substrate. In order to limit the accumulation of glycogen during the fed-batch phase. The person skilled in the art will know how to define the feed rates allowing to have carbonaceous substrate contents in the fermentation must lower than 5 g/L, preferably lower than 1 g/L, more preferentially lower than 0.1 g/L.

The growth phase is followed by a maturation phase. During this maturation phase a decrease in dry mass per liter of must is observed due to the consumption of reserve sugars accumulated during growth, in particular glycogen. At the same time, an increase in the amount of phycocyanin per gram of dry matter can be observed. The same is true for the protein content. This maturation process allows a biomass with a low glycogen content to be obtained.

According to a first embodiment of the invention, the culture is fed with a maturation feed medium that does not comprise a carbon source. It is understood that the absence of carbon source is observed also in case the maturation feed medium comprises detectable traces of carbon source.

According to a preferred embodiment of the invention, the weaning is total, i.e., the cells are no longer fed with culture medium, the cells initiating their maturation by feeding on the residual elements of the fermentation must and their cell reserves.

Advantageously, the biomass obtained comprises less than 20% of glycogen by mass in relation to the mass of dry matter (% DM), preferably less than 15% DM, more preferably less than 10% DM.

The maturation time can be more or less long depending on the temperature of culture. The closer the temperature is to the optimum temperature for growth, the shorter the maturation phase will be.

It is also possible to carry out cultures in fed-batch mode by carrying out the growth and maturation phases concomitantly, imposing a lower growth rate through the carbon source supply, thus limiting the accumulation of glycogen in the strain. The growth rate for this maturation is determined according to the maximum growth rate of the strain, which the person skilled in the art will be able to determine. This growth rate should be less than 80% of the maximum growth rate of the strain, preferentially less than 70% of the maximum growth rate of the strain, more preferentially less than 50% of the maximum growth rate of the strain.

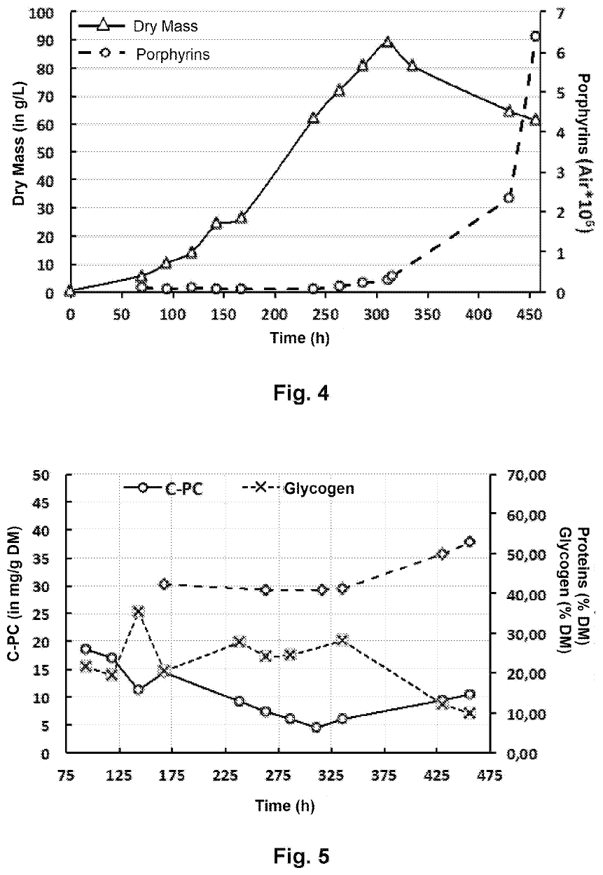

The process in accordance with the invention in fed-batch mode makes it possible to obtain fermentation musts comprising at least 70 g/L DM (FIG. 4), and a biomass with a PC content, in particular C-PC, of at least 10 mg/g DM (FIG. 5) and a protein content of at least 40% of the DM (FIG. 5).

Continuous Culture.

Continuous growth with substrate limitation has already been described by Sloth et al., 2006 with detection levels below 0.05 g/L, which the authors consider to be the analytical limit of detection. In this case, the feed medium is continuously supplied to the culture but is consumed almost instantaneously by the cells and therefore is not quantifiable. The aim of the authors in this article is to limit the inhibition of PC synthesis linked to a significant glucose concentration in the medium, glucose being known to repress PC synthesis. The PC contents under these conditions are very low, about 28 mg/g dry matter, with a biomass content of about 5 g/L dry matter.

The object of the invention is to carry out a continuous culture to increase the biomass and PC productivity compared with a fed-batch culture. It is possible, by the process in accordance with the invention, to reach a dry mass of 65-70 g/L, or even more, and a PC content comprised between 25 and 90 mg/g DM, or even more.

According to a first embodiment of the invention, the maturation phase is implemented by transferring a portion of the must from the continuous culture into a tank without nutrient supply. As with the fed-batch culture mode described above, there is a concomitant decrease in glycogen content and an increase in phycocyanin and protein content.

The person skilled in the art will know how to determine the time necessary for maturation according to known parameters such as the temperature at which the biomass is maintained without feeding and according to the desired objective. This maturation time will be at least 12 h, preferentially at least 48 h, more preferentially at least 72 h.

The growth and maturation steps can also be implemented simultaneously by imposing a reduced growth rate via the flow rate of the feed medium. Advantageously, the growth rate is less than 0.06 h-1, preferentially less than 0.03 h-1, more preferentially less than 0.015 h-1.

Advantageously, the growth rate is less than 80% of the maximum growth rate of the strain, preferentially less than 60% of the maximum growth rate of the strain, more preferentially less than 40% of the maximum growth rate of the strain. In FIG. 7, after 400 h of culture, the growth rate was reduced to a value below 80% of the maximum growth rate, thereby increasing the PC and protein content in the biomass and reducing the glycogen content, compared with the initially imposed growth rate.

The carbon source content ensures that a dry matter content of at least 65 g/L, or even at least 70 g/L, more particularly at least 80 g/L, is obtained.

The biomass thus obtained with separate or simultaneous maturation has a glycogen content of less than 20%, advantageously less than 15% or even less than 10%. The biomass obtained has a protein content of at least 45% of the DM, advantageously at least 50%.

The phycocyanin content, in particular C-PC, will be at least 20 mg/g DM, advantageously from 25 to 50 mg/g DM. With certain carbon sources, such as polyols, C-PC contents of more than 50 mg/g DM can be achieved.

Porphyrin Production in the Fermentation Must (b.1).

Several studies have shown that several key steps in the phycocyanin and chlorophyll synthesis pathway are induced by light and others repressed in the presence of organic substrate in the medium (Foley et al., 1982; Brown et al., 1982; Troxler et al., 1989; Rhie and Beale, 1994; Stadnichuck et al., 1998). According to the literature, heterotrophic culture leads to the excretion of coproporphyrin in the growth medium and causes a reduction of pigment contents in the cell (phycocyanobilin and chlorophyll) during the transition from coproporphyrinogen III to protoporphyrinogen IX. Under mixotrophic conditions, light induces both the biosynthesis of photosynthetic pigments and the excretion of porphyrins into the environment. The transition from one form of porphyrin to another is sometimes done by spontaneous reaction, so it is possible to find several forms of porphyrins in the growth medium (Brown et al., 1982; Stadnichuck et al., 1998).

Contrary to what is described in the literature, the implementation of the process in accordance with the invention does not lead to porphyrin excretion as long as a source of organic carbon, in particular glucose, glycerol, lactose, or sucrose, is present in the medium. Porphyrins are only detected during the maturation phase (without organic carbon in the medium) in both fed-batch and continuous cultures. When organic substrate is added to the medium after a maturation phase, a re-consumption of porphyrins by the cells can be observed and a return to non-detectable levels of these porphyrins in the fermentation juice.

After a maturation phase in accordance with the invention, a fermentation must is obtained comprising a biomass with a low glycogen content, rich in protein and PC, as defined above, and a juice containing porphyrins.

These porphyrins produced by URA, in particular by Galdieria sulphuraria, are natural chelators that can be used for example for treatments against nematodes (US 2006/0206946).

Some molecules such as Protoporphyrin IX could also be of interest in the medical field for cancer treatments by phototherapy (Huang et al., 2015) The invention therefore relates to a process which includes a step of recovering the fermentation juice and extracting porphyrins from this juice.

The fermentation juice is recovered by all usual biomass separation methods, in particular by centrifugation (plate centrifuge or sedicanter), well known to the skilled person, or by filtration (plate filter, filter press, ceramic or organic tangential filtration).

Porphyrins can be extracted by usual methods, like chromatography (affinity or size-exclusion chromatography).

The extracted porphyrins can be purified and then packaged for further use, in particular in therapy.

Grinding of Biomass (c.1).

To extract phycocyanins and/or proteins from the biomass, it is necessary to perform a cell lysis that will release the desired products. URA and in particular Galdieria sulphuraria have a very resistant cell wall which makes lysis by usual methods difficult unless the operating conditions are such as to degrade the desired phycocyanins.

The yield of recovered product, phycocyanins and/or proteins, does not only depend on the content of product in the biomass, but also on the capacity to extract the maximum from this biomass. This extraction capacity will depend on the efficiency of the cell lysis, but also on its implementation under conditions that do not lead to substantial degradation of phycocyanins.

Grinding is based on two major issues: the grinding rate and the heat generated by mechanical friction. In the case of phycocyanin, this heat control is even more important because it is a thermosensitive molecule. Tests were performed with a Bertoli type high-pressure homogenizer under the conditions described in Example 7. Grinding with a high-pressure homogenizer requires multiple passes to achieve a consistent lysis rate. The passage of a Galdieria sulphuraria biomass did not allow a lysis rate higher than 40% to be obtained despite 3 successive passages. At each passage, an increase in temperature could be observed until a biomass with a temperature around 70° C. was reached with a green-brown color where the majority of phycocyanin was degraded.

The invention therefore relates to a process for lysing URA cells, in particular Galdieria sulphuraria, characterized in that the lysis is carried out by grinding with a ball mill while maintaining the URA biomass during the grinding at a temperature below 50° C.

The invention consists in regulating the temperature of the biomass during grinding, inside the grinding chamber, so that it does not exceed 50° C., preferentially 47° C., more preferentially 40° C. and less. This temperature control can be done by a water cooling system of the mill jacket or by injecting into the mill a biomass previously cooled to temperatures below 20° C.

The grinding process in accordance with the invention can be applied to biomass regardless of the way it is obtained (fermentation mode and isolation). It is particularly suitable and preferable for biomass obtained by the cultivation process in accordance with the invention described above with a reduced glycogen content.

The invention also relates to the lysed biomass thus obtained.

The inventors have found that the biomass ground in accordance with the invention provides better protein digestibility than unground biomass. This improvement in digestibility has been demonstrated by in vitro digestibility tests (Boisen and Fernandez, 1995).

| Samples | Digestibility | Total Protein |
|---|---|---|
| Spirulina | 79.3 (±2) % | 68.2 (±2) % |
| G. sulphuraria (ground) | 92.2 (±2) % | 63.4 (±1.9) % |
| G. sulphuraria (unground) | 66 (±2) % | 63.9 (±1.9) % |

The invention therefore relates to a ground URA biomass and in particular to a Galdieria sulphuraria biomass obtainable by the grinding process in accordance with the invention.

The invention relates in particular to a ground biomass of Galdieria sulphuraria of the composition described below.

| Nutritional Factors | | |
|---|---|---|
| Energy Value | 394 (±22) | kcal/100 g |
| Protein | 64.8 (±9.3) | g/100 g |
| Lipids | 6.15 (±0.5) | g/100 g |
| Fibers | 7.65 (±5.16) | g/100 g |
| Carbohydrates | 16.1 (±2.2) | g/100 g |
| Ashes | 4.1 (±0.6) | g/100 g |
| Humidity | 4.1 (±0.6) | g/100 g |
| Phycocyanin | 7 (±0.3) | g/100 g |

The amino acid composition is given in the following table.

| g/100 g | Spirulina | Galdieria sulphuraria |
|---|---|---|
| Tryptophan | 0.84 | 0.76 |
| Threonine | 2.78 | 3.06 |
| Aspartic acid | 5.47 | 4.73 |
| Serine | 2.74 | 3.54 |
| Lysine | 2.7 | 3.45 |
| Valine | 3.48 | 3.15 |
| Proline | 2.04 | 2.17 |
| Alanine | 4.11 | 3.44 |
| Phenylalanine + Tyrosine | 5 | 5.55 |
| Isoleucine | 3.17 | 2.6 |
| Glycine | 2.85 | 2.30 |
| Arginine | 3.6 | 3.14 |
| Leucine | 5.02 | 4.1 |
| Histidine | 1.09 | 0.86 |
| Glutamic Acid | 8.02 | 7.41 |
| Methionine + cysteine | 2.19 | 1.88 |

The lipid composition is as follows:

| Fatty acids | % total lipids | g/100 g |
|---|---|---|
| C14:0 Myristic acid | 2.6 | 0.16 |
| C16:0 Palmitic acid | 27.9 | 1.7 |
| C18:0 Stearic acid | 8.8 | 0.54 |
| C18:1 (n-9c) Oleic acid | 33.5 | 2.1 |
| C18:2 (n-6c) Linoleic acid (LA) ω6 | 19.3 | 1.18 |
| C18:3 (n-3) α-linolenic acid (ALA) ω3 | 1.8 | 0.1 |
| Ratio ω6/ω3 | 10.32 | |

The invention also relates to the use of this ground biomass as a food supplement or food for human or animal consumption.

Extraction of Phycocyanin (d1).

The invention also relates to a process for extracting phycocyanin from a biomass of lysed URA cells, in particular Galdieria sulphuraria, characterized in that it comprises successive washes in amounts of water representing in total less than 4 times, preferably from 2 to 3 times, more preferentially about 3 times the total volume of lysed biomass.

This lysed biomass comprises a suspension of insoluble cell residues in an aqueous solution comprising various cell extracts solubilized following cell lysis, including phycocyanins.

The lysed biomass advantageously comprises a dry matter of at least 2%, preferentially of at least 5%, more preferentially of at least 7%.

The total volume of water (Vw) required for extraction is calculated as a function of the volume of lysed biomass to be treated (Vb) and will represent up to 4 times this volume (Vw/Vb is less than or equal to 4). Of course, it is possible to implement the invention with a larger total volume of water, but the economy of the process remains less interesting because of the volumes of water to be treated afterwards to recover the phycocyanin.

This total volume of water is then divided into several fractions which will be used to extract the phycocyanin by successive passages on the biomass, the number of fractions (n) being at least 2, preferably at least 3. The person skilled in the art can plan to perform the extraction with more than 3 fractions of water, while taking into account all the economic parameters of the implementation of the process, such as the cost price of the immobilization of the equipment and the repetition of the handling of the biomass. Preferably, the number of fractions is 3.

According to a first embodiment of the invention, the fractions have respective volumes different from each other. According to another embodiment of the invention, all fractions have the same volume equal to Vw/n.

The person skilled in the art will know how to determine the number of fractions and the respective volume of each fraction in order to optimize the phycocyanin preparation process that he or she will implement.

Successive washes of the pelleted insoluble elements are required to extract an appropriate amount of phycocyanin from the biomass. After several washes, the proportions of C-PC and APC in the pellet are reversed. It is clear from FIG. 16 that it is better to extract phycocyanin with successive small volumes of water rather than with an equivalent large volume of water (FIG. 16).

On the y-axis and starting from the left of the diagram, there are three blocks S1, S2 and S3 which correspond to 3 successive extractions made with 3 fractions of water of equal volume, the total volume of water Vw being 1 time the volume of biomass Vb for the first block (½ serial dilution), 2 times for the second block (⅓ serial dilution) and 3 times for the third block (¼ serial dilution). Bar S1 gives the PC value extracted by the first extraction. Bar S2 gives the cumulative value of PC extracted by the first and second extraction. Bar S3 gives the cumulative value for the 3 fractions used successively. There are then 5 single extraction trials with different volumes of water, from 5× to 12×.

From FIG. 16, it can be seen that a larger initial volume of water in the first extraction gives better results up to a certain limit (5× to 12×). Using the serial extraction method, a real gain can be seen in terms of extraction efficiency and reduction in water usage. For example, 3 series of extractions give better results than a single extraction and reduce the total amount of water used by at least a factor of 2.

The wash waters recovered from each successive extraction including phycocyanin can be treated separately to recover the phycocyanin or assembled before this recovery.

The extraction process in accordance with the invention is suitable for any lysed biomass of URA, in particular Galdieria sulphuraria, irrespective of the culture method employed for biomass production and the method employed for cell lysis. Preferentially, the extraction method in accordance with the invention is particularly suitable for biomass with low glycogen contents obtained by the process in accordance with the invention described above and/or for biomass lysed by the grinding method in accordance with the invention defined above.

The resulting phycocyanin solution is usually treated to isolate the phycocyanin. Methods for recovering phycocyanin from an aqueous solution are well known to the skilled person. Particular mention may be made of the acid precipitation described in patent application WO 2018/178334.

It can also be isolated by selective precipitation which consists in adjusting the pH of the initial solution to a value chosen in a range of pH values in which the phycocyanin is less soluble (also called instability range) and in concentrating the phycocyanin in the solution to promote its precipitation, then in recovering the precipitated phycocyanin. This range of instability goes in particular from pH 4.4 to 5.5 for acid-resistant phycocyanins produced by Galdieria sulphuraria. Of course, the person skilled in the art will know how to determine such a range of instability for other phycocyanins produced by other URA by simple experimentation. Such a method is described in particular in patent application FR 1900278 filed on Jan. 11, 2019.

Before recovery of the phycocyanin, the aqueous solution can also be treated to lower its glycogen content by enzymatic degradation of glycogen. The traces of these polysaccharides likely to be carried away with the precipitation of phycocyanins, already low, are even more reduced when the polysaccharides are lysed into low-molecular-weight polysaccharides even more soluble. Moreover, when the concentration step is performed by tangential filtration, the low-molecular-weight polysaccharides are eliminated with the other small molecules in solution, which favors the obtaining of a solution with an even higher phycocyanin content. In particular, enzymatic lysis of glycogen is carried out at a pH of 5 or less, preferably about 4.5, at room temperature. These temperature and pH conditions are particularly suitable for preserving the phycocyanin during the enzymatic reaction. Enzymes active under acidic pH and room temperature conditions are selected from enzymes known to have α1-4 glucuronidase, α1-4 glucosidase (or alpha-glucosidase) activity. Particular mention may be made of pectinases known to degrade pectin and in particular pectinases extracted from filamentous fungi such as Aspergillus, more particularly pectinases extracted from Aspergillus aculeatus, such as the enzymes marketed under the name Pectinex® by the company Novozymes. Enzymatic lysis of glycogen could also be performed with an α1-6 glucosidase in addition to the α1-4 glucuronidase or α1-4 glucosidase. α1-6 Glucosidases active under the pH and temperature conditions set forth above are also known to the skilled person. In particular, these are pullulanases known to hydrolyze α1-6 glycosidic bonds of pullulan, in particular known to remove starch branches. These are generally enzymes extracted from bacteria, particularly from the genera Bacillus. U.S. Pat. Nos. 6,074,854, 5,817,498 and WO 2009/075682 describe such pullulanases extracted from Bacillus deramificans or Bacillus acidopullulyticus. Commercially available pullulanases are also known, in particular under the names “Promozyme D2” (Novozymes), “Novozym 26062” (Novozymes) and “Optimax L 1000” (DuPont-Genencor). It will be noted that pullulanase/alpha-amylase mixtures are described in the prior art, but in particular to produce glucose syrup from starch (US 2017/159090). The person skilled in the art will know how to determine the appropriate reaction conditions to best reduce the amounts of glycogen depending on the initial glycogen content in the solution to be treated, the amount of enzymes employed and the purity sought for the phycocyanin produced. Such a method is described in particular in patent application FR 1900278 filed on Jan. 11, 2019.

The recovered phycocyanin can then be purified by methods known to the skilled person, such as diafiltration.

The phycocyanin obtained by the extraction process in accordance with the invention has a purity index of at least 2, preferably at least 3, or even higher than 4. This purity index is measured by absorbance measurement with the method described by Moon et al. (2014).

Advantageously, the phycocyanin obtained is a phycocyanin which has a glycogen/phycocyanin ratio (on a dry weight basis) lower than 6, advantageously lower than 4, preferably lower than 3, more preferentially lower than 2.5, even more preferentially lower than 1.

The invention also relates to the use of the phycocyanins obtained as colorants, in particular as food colorants. It also relates to foodstuffs, solid or liquid, in particular beverages which comprise a phycocyanin obtained by the extraction process in accordance with the invention.

The solid residues remaining after washing are also recovered. It is a biomass residue enriched in proteins which can also be used for the preparation of food supplements or food for human or animal consumption.

According to a particular embodiment, washing the lysed biomass comprises acidification of the biomass suspension to a pH of less than or equal to 5. The residual biomass obtained after phycocyanin extraction comprises at least 60% protein based on dry matter, and at least a total sugar content of less than 20% based on dry matter and/or a glycogen content of less than 10% based on dry matter and/or a fat content of at least 5% based on dry matter. Such a method for recovering a protein-enriched biomass is described in particular in patent application FR 1857950 filed on Sep. 5, 2018.

### EXAMPLES

Materials and Methods.

Strain.

Galdieria sulphuraria UTEX #2919, also called Cyanidium caldarium.

Growing conditions in fed-batch mode.

The cultures are carried out in bioreactors of 1 to 2 L of useful volume with dedicated liquid handlers and supervision by computer station. The pH of the culture is regulated by adding base (ammonia solution 14% NH3 w/w) and/or acid (4N sulfuric acid solution). The culture temperature is set at 37° C. Stirring is done by 2 stirring spindles: 1 Rushton turbine with 6 straight blades positioned at the lower end of the stirring shaft above the sparger and 1 HTPG2 three-bladed propeller placed on the stirring shaft. The pressure of dissolved oxygen in the liquid phase is regulated in the medium throughout the culture by the speed of rotation of the stirring shaft (250-1800 rpm), the flow of air and/or oxygen. The regulation parameters, integrated in the supervision automaton, allow to maintain a partial pressure of dissolved oxygen in the liquid phase comprised between 5 and 30% of the saturation value by the air in identical conditions of temperature, pressure and composition of the medium. The culture time was comprised between 50 and 300 hours.

Continuous Mode Culture Conditions.

The cultures are carried out in reactors of 1 to 2 L of useful volume with dedicated liquid handlers and supervision by computer station. The pH of the culture is regulated by adding base (ammonia solution 14% (w NH3/w) and/or acid (4N sulfuric acid solution). The culture temperature is set at 37° C. Stirring is done by two stirring spindles: 1 Rushton turbine with 6 straight blades positioned at the lower end of the stirring shaft above the sparger and 1 HTPG2 three-bladed propeller placed on the stirring shaft. The pressure of dissolved oxygen in the liquid phase is regulated in the medium throughout the culture, by the speed of rotation of the stirring shaft (250-1800 rpm), the flow of air and/or oxygen. The regulation parameters, integrated in the supervision automaton, allow to maintain a partial pressure of dissolved oxygen in the liquid phase comprised between 5 and 30% of the saturation value by the air in identical conditions of temperature, pressure and composition of the medium. The culture time was between 50 and 300 hours. The feed rate of the continuous fermenter was adjusted so that at no time was the carbon source detected in the culture medium.

Feed Medium.

For the fed-batch or continuous mode feed medium, the amount of carbon source is adjusted to the target dry weight at the end of fed-batch or 100 g/L dry weight for continuous culture. All other elements of the medium are added in the proportions used for the starter medium defined in the examples.

Culture Monitoring.

Growth is monitored by measuring the dry mass (filtration on GF/F filter, Whatman, then drying in oven at 105° C. for 24 h minimum before weighing).

Determination of Porphyrins.

The determination of organic acids was performed by HPLC (Shimatsu) in H₂SO₄ isocratic mode (5 mM) and RI (Refractive Index) detection.

Determination of PC.

Estimation of phycocyanin content per gram of dry matter was performed at different culture times using the process described by Moon and collaborator [Moon et al., Korean J. Chem. Eng., 2014, 1-6]

Determination of Glycogen.

Estimation of glycogen content per gram of dry matter was performed at different culture times using the extraction process described by Martinez-Garcia and collaborator [Martinez-Garcia et al., Int J Biol Macromol. 2016; 89:12-8].

### Example 1: Fed-Batch Fermentation with Maturation Phase on Glycerol

Culture Medium.

Starter: 30 g/L glycerol, 8 g/L (NH₄)₂SO₄, 250 mg/L KH2PO4, 716 mg/L MgSO₄, 44 mg/L CaCl₂, 2H₂O, 0.2843849 g/L K₂SO₄, 0.07 g/L FeSO₄, 7H₂O, 0.01236 g/L Na₂EDTA, 0.00657 g/L ZnSO₄, 7H₂O, 0.0004385 g/L CoCl₂, 6H₂O, 0.00728 g/L MnCl₂, 4H₂O, 0.005976 g/L (NH₄)₆Mo₇O₂₄, 4H₂O, 0.005976 g/L CuSO₄, 5H₂O, 0.00016 g/L NaVO₃, 0.01144 g/L H₃BO₃, 0.00068 g/L Na₂SeO₃.

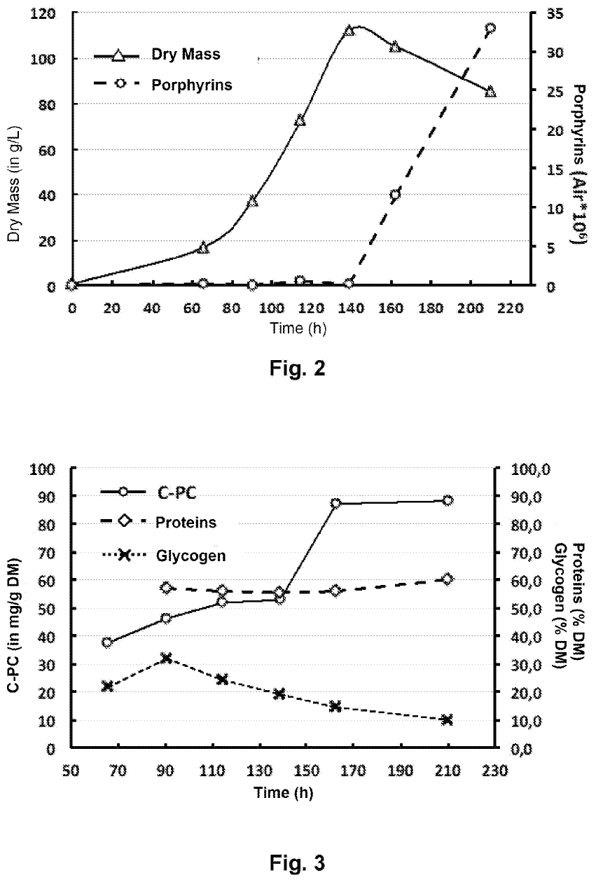

The results are presented in FIGS. 2 and 3.

### Example 2: Fed-Batch Fermentation on Glucose with Maturation Phase

Culture Medium.

Starter: 30 g/L glucose, 8 g/L (NH₄)₂SO₄, 250 mg/L KH2PO4, 716 mg/L MgSO₄, 44 mg/L CaCl₂, 2H₂O, 0.2843849 g/L K₂SO₄, 0.07 g/L FeSO₄, 7H₂O, 0.01236 g/L Na₂EDTA, 0.00657 g/L ZnSO₄, 7H₂O, 0.0004385 g/L CoCl₂, 6H₂O, 0.00728 g/L MnCl₂, 4H₂O, 0.005976 g/L (NH₄)₆Mo₇O₂₄, 4H₂O, 0.005976 g/L CuSO₄, 5H₂O, 0.00016 g/L NaVO₃, 0.01144 g/L H₃BO₃, 0.00068 g/L Na₂SeO₃.

The results are presented in FIGS. 4 and 5.

### Example 3: Fed-Batch Fermentation on Sucrose with Maturation Phase

Culture Medium

Starter: 30 g/L sucrose, 8 g/L (NH₄)₂SO₄, 250 mg/L KH2PO4, 716 mg/L MgSO₄, 44 mg/L CaCl₂, 2H₂O, 0.2843849 g/L K₂SO₄, 0.07 g/L FeSO₄, 7H₂O, 0.01236 g/L Na₂EDTA, 0.00657 g/L ZnSO₄, 7H₂O, 0.0004385 g/L CoCl₂, 6H₂O, 0.00728 g/L MnCl₂, 4H₂O, 0.005976 g/L (NH₄)₆Mo₇O₂₄, 4H₂O, 0.005976 g/L CuSO₄, 5H₂O, 0.00016 g/L NaVO₃, 0.01144 g/L H₃BO₃, 0.00068 g/L Na₂SeO₃.

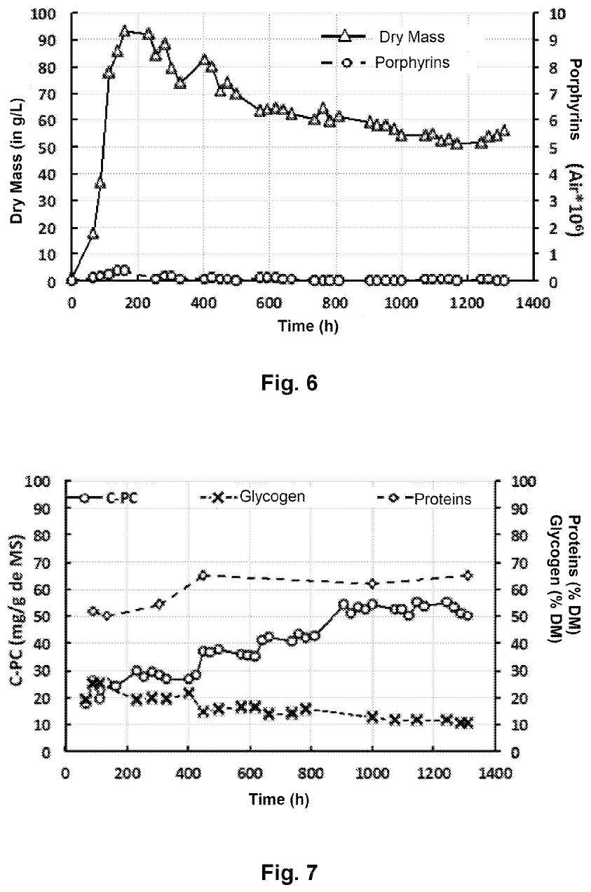

The results are shown in FIGS. 6 and 7.

### Example 4: Fed-Batch Fermentation on Milk Permeate with Maturation Phase

Culture Medium.

Starter: 30 g/L milk permeate, 8 g/L (NH4)₂SO₄, 716 mg/L MgSO₄, 0.07 g/L FeSO₄, 7H₂O, 0.01236 g/L Na₂EDTA, 0.00657 g/L ZnSO₄, 7H₂O, 0.0004385 g/L CoCl₂, 6H₂O, 0.00728 g/L MnCl₂, 4H₂O, 0.005976 g/L (NH₄)₆Mo₇O₂₄, 4H₂O, 0.005976 g/L CuSO₄, 5H₂O, 0.00016 g/L NaVO₃, 0.01144 g/L H₃BO₃, 0.00068 g/L Na₂SeO₃.

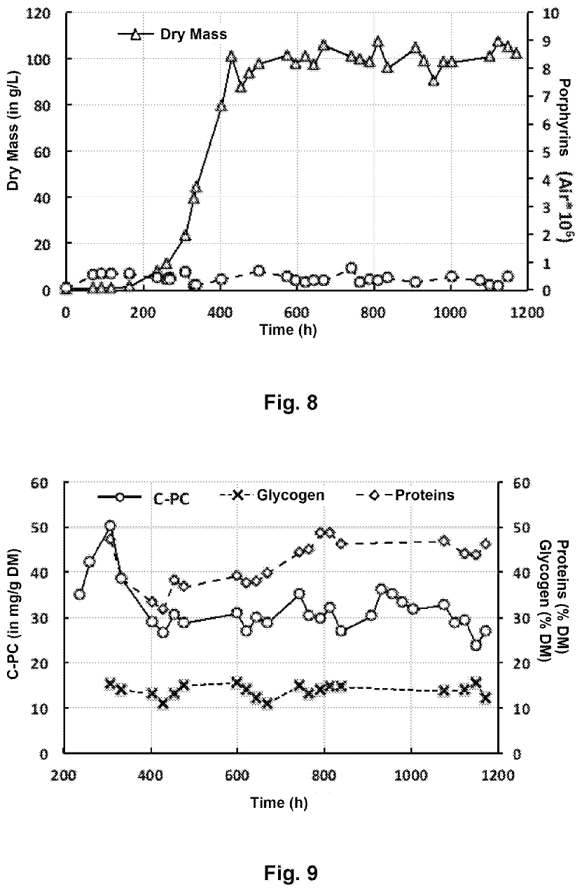

The results are presented in FIGS. 8 and 9.

### Example 5: Continuous Culture of a Galdieria Strain on Glycerol

Culture Medium.

Starter: Starter: 30 g/L glycerol, 8 g/L (NH₄)₂SO₄, 250 mg/L KH2PO4, 716 mg/L MgSO₄, 44 mg/L CaCl₂, 2H2O, 0.2843849 g/L K₂SO₄, 0.07 g/L FeSO4, 7H2O, 0.01236 g/L Na2EDTA, 0.00657 g/L ZnSO4, 7H2O, 0.0004385 g/L CoCl2, 6H2O, 0.00728 g/L MnCl2, 4H2O, 0.005976 g/L (NH4)6Mo7O24, 4H2O, 0.005976 g/L CuSO4, 5H₂O, 0.00016 g/L NaVO3, 0.01144 g/L H3BO3, 0.00068 g/L Na2SeO3.

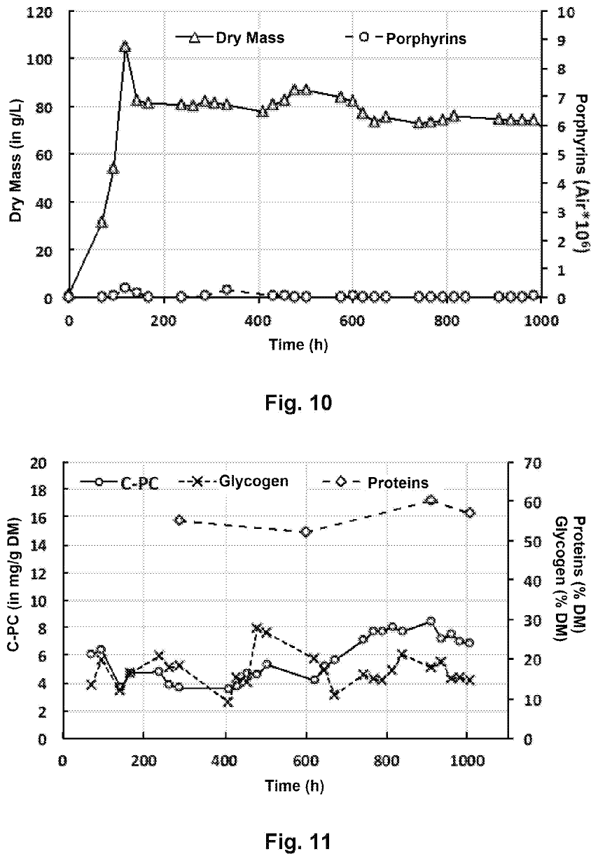

The results are shown in FIGS. 10 and 11.

### Example 6: Continuous Culture of a Galdieria Strain on Milk Permeate

Culture Medium.

Starter: Starter: 30 g/L milk permeate, 8 g/L (NH₄)₂SO₄, 716 mg/L MgSO₄, 0.2843849 g/L K₂SO₄, 0.07 g/L FeSO₄, 7H₂O, 0.01236 g/L Na₂EDTA, 0.00657 g/L ZnSO₄, 7H₂O, 0.0004385 g/L CoCl₂, 6H₂O, 0.00728 g/L MnCl₂, 4H₂O, 0.005976 g/L (NH4)₆Mo7O₂₄, 4H₂O, 0.005976 g/L CuSO₄, 5H₂O, 0.00016 g/L NaVO₃, 0.01144 g/L H₃BO₃, 0.00068 g/L Na₂SeO₃.

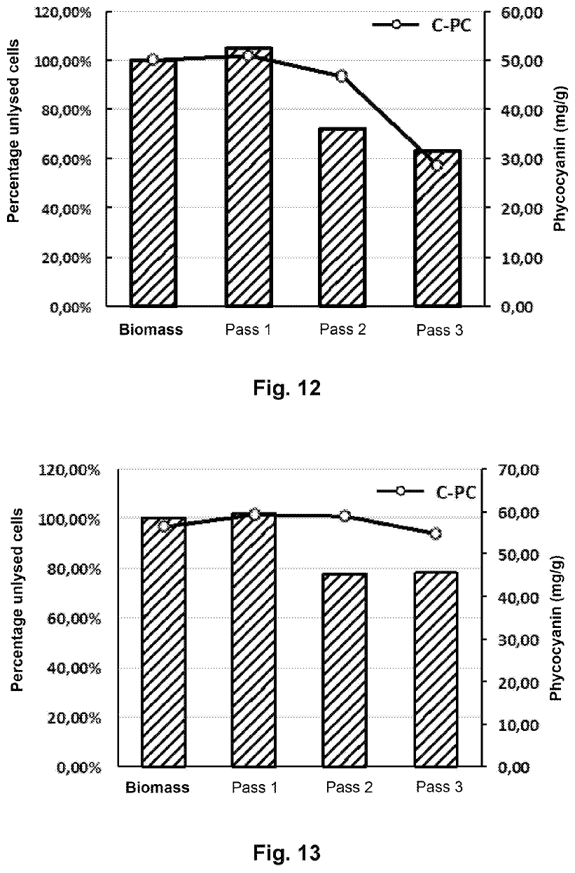

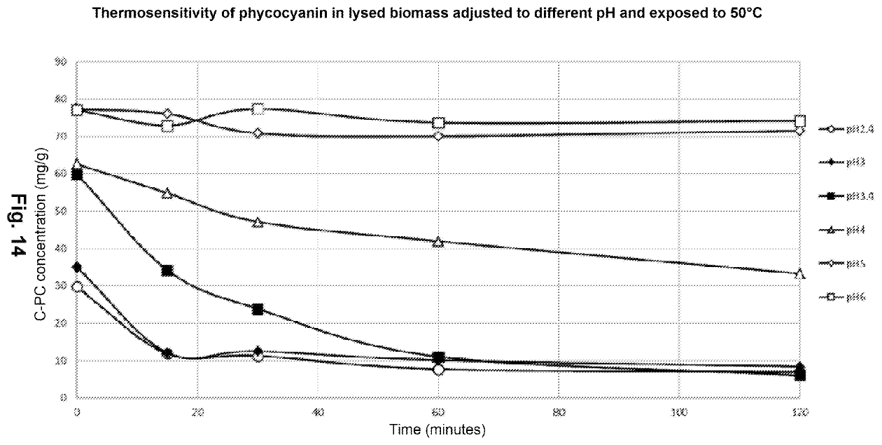

The results are shown in FIGS. 12 and 13.

### Example 7: HHP Milling without Cooling of Biomass

Procedure.

A biomass from a continuous culture is washed by successive centrifugations then concentrated to a concentration of 1.4.10¹⁰ cells/mL. A volume of 1 L of biomass is then cooled to 16° C. before undergoing 3 successive homogenizations at 1200 bar on a Bertoli Atomo homogenizer, without cooling between each series. For each of them, the temperature of the biomass, the cell lysis by counting with Malassez cells and the concentration of phycocyanin in the biomass are monitored.

The measured grinding temperatures for the 3 successive homogenizations are 46.7° C., 57.6° C. and 67° C., respectively.

The percentages of unlysed cells and phycocyanin contents are given in FIG. 12.

### Example 8: HHP Milling with Biomass Cooling

Procedure.

A biomass from a continuous culture is washed by successive centrifugations and then concentrated to a concentration of 2.10¹⁰ cells/mL. A volume of 1 L of biomass is then cooled to 16° C. before undergoing 3 successive homogenizations at 1200 bar on a Bertoli Atomo homogenizer. Between each homogenization, the temperature of the biomass is brought back to 16° C. In the same way, the temperature of the biomass, the cell lysis by counting with Malassez cells and the concentration of phycocyanin in the biomass are monitored.

The biomass temperatures measured at the beginning and end of the milling process are as follows.

| | | | | |
|---|---|---|---|---|
| T° at grinding start | 16° C. | 16.1° C. | 15.4° C. | 14.4° C. |
| T° at grinding end | 42° C. | 45.2° C. | 47.7° C. | 46° C. |

The percentages of unlysed cells and phycocyanin contents are given in FIG. 13.

It was also found that the more acidic the pH of the ground biomass, the more sensitive the phycocyanin is to degradation by heat. It is therefore preferable to adjust the pH of the cells to between 5 and 7 before grinding them, regardless of the grinding method if it is accompanied by release of heat.

### Example 9: Thermosensitivity of Phycocyanin in the Biomass

Procedure.

Biomass from a continuous culture is washed by successive centrifugations and concentrated to a dry matter of 150 mg/g before being ground by ball mill (WAB, multilab) under conditions allowing preservation of pigment and a lysis rate of 90%. Lysate samples are adjusted to pH 2.4 to 6 and kinetics from 0 to 120 minutes are performed at different temperatures ranging from 50 to 70° C. For each time a quantification of phycocyanin is performed.

The results are shown in FIG. 14.

### Example 10: Effect of Bead Size on Grinding and Temperature Control

Procedure.

Galdieria sulphuraria cells are centrifuged for 5 min at 20 000 g and then re-suspended in 10 mM Tris-CI buffer pH 7. A cell aliquot ⅓ of the volume of a 2 mL Safelock Eppendorf tube is filled with this suspension, another ⅓ with ceramic beads of the tested diameter (Netzsch 0.8 mm; 0.6 mm; 0.3 mm; Plus 0.2 mm; Nano 0.2 mm; Plus 0.1 mm; and 0.05 mm).

Tubes are placed in a TissueLyser II apparatus (Qiagen) and shaken for 2 min at 30 Hz. Lysis rate is calculated by Malassez cell count compared with the control tube containing no beads.

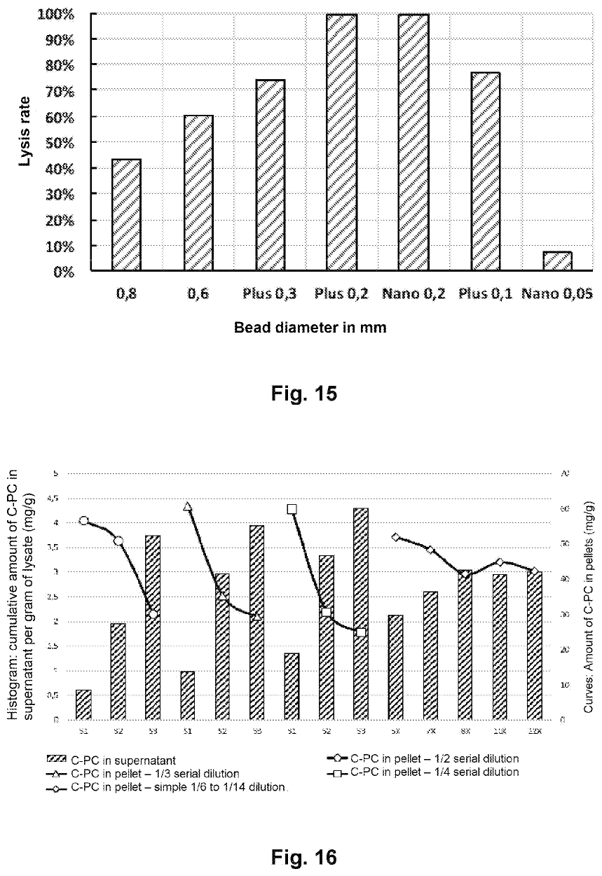

The results are shown in FIG. 15.

It can be seen that the diameter of the beads greatly affects the grinding efficiency. As the bead diameter decreases, the lysis rate increases until it reaches an optimum for beads with a diameter of 0.2 mm. Below this diameter the lysis efficiency decreases again until it reaches the lowest rate for beads with a diameter of 0.05 mm.

Grinding rates close to 100% can be achieved with larger beads if the grinding time is increased. However, increasing the grinding time results in a rise in temperature in the grinding chamber with the imposed ball mill feed rate. This temperature increase is at the expense of the phycocyanin content of the lysed biomass.

### Example 11: Effect of Chamber Filling Rate on Grinding Rate

Procedure.

The cells are ground in a Multilab model ball mill from WAB. The grinding chamber is filled with ceramic beads of 0.8 mm diameter at 50% and 65%. The 65% filling rate is the maximum filling rate. The grinding module speed and flow rate are identical in both cases. The lysis rate at the milling exit is calculated by counting in the Malassez cell compared with the unmilled input biomass.

It can be seen that the best lysis rate is obtained when the chamber is at its maximum filling rate recommended by the manufacturer, i.e., 65%. When the filling rate is lower than 65% the lysis rate decreases.

### Example 12: Effect of Cell Concentration on Grinding Rate and Temperature Control

Procedure.

The cells are ground in a Multilab model ball mill from WAB. The grinding chamber is filled with ceramic beads of 0.8 mm diameter at a rate of 65%. The grinding module speed and flow rate are identical in all cases. The lysis rate at the milling outlet is calculated by Malassez cell count compared with the unmilled input biomass.

It can be seen that for the same grinding parameters (grinding module speed, feed rate, chamber filling rate, bead diameter) the lysis rate obtained was equivalent whatever the cell concentration in the product to be ground (biomass at 10%, 20% or 30% dry matter). However, it should be noted that the higher the dry mass of the input product, the higher the temperature of the lysate leaving the mill. In order to increase the productivity of the milling step by increasing the input dry mass, it is also necessary to provide cooling capacities adapted to maintaining a lysate temperature below 45° C.

### Example 13: Estimation of Flow Rates on an Industrial Ball Mill

Materials and Methods.

Strain: Galdieria sulphuraria (also called Cyanidium caldarium) UTEX #2919

Procedure.

The cells are ground in a Multilab model ball mill from WAB. The grinding chamber (600 mL) is filled with ceramic beads of 0.8 mm diameter at a rate of 65%. The speed of the grinding module and the feed rate are identical in both cases. The lysis rate at the mill outlet is calculated by Malassez cell count compared with the unmilled biomass inlet. For a grinding rate of 95-100% the flow rate applied under these conditions is between 1 and 2 liters per hour.

An extrapolation of these flow rates was made using the results obtained in Example 10.

| | Multilab (0.6 mL) | AP60 (60 L) |
|---|---|---|
| Bead size | Supply L/h | Supply L/h |
| 0.8 mm | 1 to 2 | 100 to 200 |
| 0.6 mm | 1.4 to 2.8 | 140 to 280 |
| 0.3 mm | 1.7 to 3.4 | 170 to 340 |
| 0.2 mm | 2.3 to 4.6 | 230 to 460 |

### REFERENCES

Bailey, R W., and Staehelin L A. The chemical composition of solated cell walls of Cyanidium caldarium. Microbiology 54, 2 (1968): 269-276.
Boisen, S. and J. A. Fernandez. 1995. Prediction of the apparent ileal digestibility of protein and amino acids in feedstuffs and feed mixtures for pigs by in vitro analyses. Anim. Feed Sci. Technol. 51:29-43.
Li S Y, Shabtai Y, and Arad S. Floridoside as a carbon precursor for the synthesis of cell-wall polysaccharide in the red microalga Porphyridium sp. (Rhodophyta). Journal of phycology 38, no 5 (2002): 931-938.
Marquardt, J, and Rhiel E. The membrane-intrinsic light-harvesting complex of the red alga Galdieria sulphuraria (formerly Cyanidium caldarium): biochemical and immunochemical characterization. Biochimica et Biophysica Acta (BBA)—Bioenergetics 1320, 2 (1997): 153 64.
Martinez-Garcia M, Stuart M C, van der Maarel M J. Characterization of the highly branched glycogen from the thermoacidophilic red microalga Galdieria sulphuraria and comparison with other glycogens. Int J Biol Macromol. 2016 August; 89:12-8.
Martinez-Garcia M, Kormpa A, van der Maarel MJEC. The glycogen of Galdieria sulphuraria as alternative to starch for the production of slowly digestible and resistant glucose polymers. Carbohydr Polym. 2017 Aug. 1; 169:75-82
Moon M, Mishra S. K, Kim C. W, Suh W. I, Park M. S, and Yang J-W. Isolation and Characterization of Thermostable Phycocyanin from Galdieria Sulphuraria. 2014. 31:1-6.
Sloth J. K, Wiebe M. G, Eriksen N. T. Accumulation of phycocyanin in heterotrophic and mixotrophic cultures of the acidophilic red alga Galdieria sulphuraria. Enzyme and Microbial Technology 38 (2006) 168-175
Huang H, Song W, Rieffel J, and Lovell J. F. Emerging applications of porphyrins in photomedicine. Frontiers in physics (2015) 3-23.
Eriksen, N T. Production of Phycocyanin—a Pigment with Applications in Biology, Biotechnology, Foods and Medicine. Applied Microbiology and Biotechnology 80, 1 (2008): 1 14.
Cruz de Jesús, Verónica, Gabriel Alfonso Gutierrez-Rebolledo, Marcela Hernandez-Ortega, Lourdes Valadez-Carmona, Angelica Mojica-Villegas, Gabriela Gutierrez-Salmeán, et German Chamorro-Cevallos. “Methods for Extraction, Isolation and Purification of C-Phycocyanin: 50 Years of Research in Review” 3, no 1 (2016).
Montalescot V, Rinaldi T, Touchard R, Jubeau S, Frappart M, Jaouen P, Bourseau P, et Marchal L. Optimization of bead milling parameters for the cell disruption of microalgae: Process modeling and application to Porphyridium cruentum and Nannochloropsis oculata. Bioresource Technology 196 (2015): 339 46.

1. An optimized process for the cultivation and valorization of unicellular red algae (URA) comprising the steps:
(a) of fermentation culture of the URA;
(b) of recovery of the biomass; and
(c) of cell lysis; and
optionally, a step (d) of extraction of valorizable products from the lysed biomass,
wherein the process comprises a step (c1) of lysis by grinding while maintaining the biomass during the grinding at a temperature below 50° C.

2. The process according to claim 1, further comprising a step (d1) of extracting the phycocyanins from the lysed biomass by successive washes in amounts of water totaling total less than 3 times the total volume of lysed biomass.

3. The process according to claim 1, further comprising one of the following steps:
(a1) of fermentation culture with a maturation phase with limited carbon source input into the culture medium, and
(b1) of extraction of porphyrins from the fermentation juice.

4. The process according to claim 1, wherein the URA belong to the genus Cyanidioschyzon, Cyanidium or Galdieria.

5. The process according to claim 4, wherein the URA belong to the genus Galdieria and to the species sulphuraria.

6. The process according to claim 1, wherein the URA are grown by fermentation in fed-batch or continuous mode.

7. The process according to claim 1, wherein the grinding temperature does not exceed 47° C.

8. The process according to claim 1, wherein the grinding temperature does not exceed 40° C.

9. The process according to claim 1, wherein the temperature is maintained by a water cooling system of the mill jacket.

10. The process according to claim 1, wherein the temperature is maintained by injecting into the mill a biomass previously cooled to temperatures below 20° C.

11. The process according to claim 1, wherein the phycocyanin is recovered from the wash solution of the lysed biomass.

12. An optimized process for the cultivation and valorization of unicellular red algae (URA) comprising the steps:
(a) of fermentation culture of the URA;
(b) of recovery of the biomass; and
(c) of cell lysis; and
optionally, a step (d) of extraction of valorizable products from the lysed biomass, wherein the process further comprises the step
(a1) of fermentation culture with a maturation phase which includes the limitation of the carbon source in the culture medium;
optionally, one or more steps selected among:
(b1) of extracting porphyrins from the fermentation juice,
(c1) of lysis by grinding while maintaining the biomass during grinding at a temperature below 50° C., and
(d1) of extraction of phycocyanins from the lysed biomass by at least 2 successive washes in amounts of water totaling less than 4 times the total volume of lysed biomass.

13. An optimized process for the cultivation and valorization of unicellular red algae (URA) comprising the steps:
(a) of fermentation culture of the URA;
(b) of recovery of the biomass; and
(c) of cell lysis; and
optionally, a step (d) of extraction of valorizable products from the lysed biomass, wherein the optimized process further comprises the step
(b1) of extraction of porphyrins from the fermentation juice,
and optionally, one or more steps selected among:
(a1) of fermentation culture with a maturation phase which includes limiting the carbon source input to the culture medium,
(c1) of lysis by grinding while maintaining the biomass during grinding at a temperature below 50° C., and
(d1) of extraction of phycocyanins from the lysed biomass by at least 2 successive washes in amounts of water representing in total less than 4 times the total volume of lysed biomass.

14. An optimized process for the cultivation and valorization of unicellular red algae (URA) comprising the steps:
(a) of fermentation culture of the URA;
(b) of recovery of the biomass; and
(c) of cell lysis; and
optionally, a step (d) of extraction of valorizable products from the lysed biomass, wherein the process further comprises the step
(d1) of extraction of phycocyanins from the lysed biomass by at least 2 successive washes in amounts of water representing in total less than 4 times the total volume of lysed biomass;
and optionally, one or more steps selected among:
(a1) of fermentation culture with a maturation phase which includes limiting the carbon source input to the culture medium,
(b1) of extraction of porphyrins from the fermentation juice, and
(c1) of lysis by grinding with the biomass being maintained during grinding at a temperature below 50° C.

15. The process according to claim 12, wherein the URA belong to the genus Cyanidioschyzon, Cyanidium or Galdieria.

16. The process according to claim 15, wherein the URA belong to the genus Galdieria and to the species sulphuraria.

17. The process according to claim 12, wherein the URA are grown by fermentation in fed-batch or continuous mode.

18. The process according to claim 12, wherein phycocyanin is recovered from the wash solution of the lysed biomass.

19. Lysed biomass obtained by the process according z claim 1.

20. Porphyrins obtained by the process according to claim 3.

21. Phycocyanin obtained by the process according to claim 2.

22. A food or food supplement, comprising the lysed biomass according to claim 19.
